(19) United States
(12) Patent Application Publication
(10) Pub. No.: US 2022/0162612 A1
(43) Pub. Date: May 26, 2022

(54) SYNTHETIC TRANSFER RNA WITH EXTENDED ANTICODON LOOP

(71) Applicant: Arcturus Therapeutics, Inc., San Diego, CA (US)

(72) Inventors: Zoya IGNATOVA, Hamburg (DE); Andrew TORDA, Hamburg (DE)

(21) Appl. No.: 17/602,251

(22) PCT Filed: Apr. 9, 2020
(86) PCT No.: PCT/EP20/60217

§ 371 Date: Oct. 7, 2021

(30) Foreign Application Priority Data
Apr. 11, 2019 (DE) 10 2019 109 664.2
Apr. 11, 2019 (LU) LU101182

(51) Int. Cl. C12N 15/113

(52) U.S. Cl. CPC C12N 15/113; C12N 2310/3513; C12N 2310/531

(57) ABSTRACT

The invention relates to a synthetic transfer RNA with an extended anticodon loop. The invention provides a synthetic suppressor transfer RNA useful for the treatment of a genetic disease like neurofibromatosis associated with a frameshift mutation. The synthetic transfer RNA comprises an extended anticodon loop having a four-nucleotide anticodon or a five-nucleotide anticodon.

The invention relates to a synthetic transfer RNA with an extended anticodon loop.

Transfer ribonucleic acids (tRNAs) are an essential part of the protein synthesis machinery of living cells as necessary components for translating the nucleotide sequence of a messenger RNA (mRNA) into the amino acid sequence of a protein. Naturally occurring tRNAs comprise an amino acid binding stem being able to covalently bind an amino acid and an anticodon loop containing a base triplet called “anticodon”, which can bind non-covalently to a corresponding base triplet called “codon” on an mRNA. A protein is synthesized by assembling the amino acids carried by tRNAs using the codon sequence on the mRNA as a template with the aid of a multi component system comprising, inter alia, the ribosome and several auxiliary enzymes.

Some diseases belonging to the group of genetic diseases are based on a change in the genetic information, e.g. a mutation in the DNA of the encoding genes. This includes single- or multiple nucleotide exchanges, deletions or insertions. In this case the mRNA transcribed from the mutated gene will also carry the altered genetic information and an aberrant, possibly non-functional protein is formed. A deletion or insertion (collectively termed indel) of one or multiple nucleotides may, for example, change the reading-frame within a coding region (i.e. the trinucleotide register in which the ribosome reads the information in mRNA) resulting in an entirely new amino acid sequence down-stream of the mutation and the production of a non-functional protein. As an example, the diseases neurofibromatosis type I (NF1) and neurofibromatosis type 2 (NF2) can be caused by a mutation of the gene coding for neurofibromin 1 (NF1) and neurofibromin 2 (NF2), respectively. The genes NF1 and NF2 are believed to function as tumor suppressors. Mutations in NF1 and NF2 occur at birth (inherited or de novo) but also in the somatic cell state. The most common mutations are frameshift mutations with deletion or insertion, ranging from 1 to 12 nucleotides, with deletions being more frequent than insertions (Pros, E., Gómez, C., Martin, T., Fábregas, P., Serra, E. and Lizaro, C. (2008), Nature and mRNA effect of 282 different NF1 point mutations: focus on splicing alterations, Hum. Mutat., 29: E173-E193. doi:10.1002/humu.20826; Ars E, Kruyer H, Morell M, et al., Recurrent mutations in the NF1 gene are common among neurofibromatosis type 1 patients, J. Med. Genet. 2003; 40:e82, doi:10.1136/jmg.40.6.e82).

Some antibiotics such as macrolides promote frameshifting, but no attempts to use them to correct frameshifting so far have been reported (Brierley, I. 2013, Macrolide-Induced Ribosomal Frameshifting: A New Route to Antibiotic Resistance, Mol. Cell 52, 613-615, doi:10.1016/j.molcel.2013.11.017; Atkins, J. F., & Baranov, P. V. (2013). Molecular biology: Antibiotic re-frames decoding. Nature, 503(7477), 478-479. Doi:10.1038/503478a).

Although still in its beginnings, gene therapy involving the introduction of corrective genetic material into the cells of a patient is becoming important for treating genetic diseases. Approaches based on correcting entities down-stream of the gene, mostly mRNA, are preferred as they do not belong to the classic gene therapy approaches, since the gene sequence (or DNA) remains unchanged. However, mRNA is intrinsically short-lived and the length of the mRNA sequences presents problems for therapeutic application. A particular mRNA may, for example, be longer than the cargo capacity of currently available vectors for gene delivery and therapy.

Compared to mRNA, tRNA molecules offer significantly higher stability and are on average 10-fold shorter, alleviating the problem of introduction into the target tissue. This has led to attempts to use tRNA in gene therapy in order to prevent the formation of a truncated protein from an mRNA with a premature stop codon and to introduce the correct amino acid instead (see, e.g., Koukuntla, R 2009, Suppressor tRNA mediated gene therapy, Graduate Theses and Dissertations, 10920, Iowa State University, http://lib.dr.iastate.edu/etd/10920; US 2003/0224479 A1; U.S. Pat. No. 6,964,859).

Natural tRNAs with an extra nucleotide in the anticodon or with a shortened codon doublet have been found in bacteria. These unusual codons are used naturally to suppress a specific frameshift position (Qian, Q., Li J-N., Zhao H., Hagervall T. G., Farabaugh P. J., Björk G. R., A New Model for Phenotypic Suppression of Frameshift Mutations by Mutant tRNAs, Mol cell 1, 471-482, 1998; O'Mahony D J, Hughes D, Thompson S, Atkins J F, Suppression of a −1 Frameshift Mutation by a Recessive tRNA Suppressor Which Causes Doublet Decoding, J Bact, 171 (7), 1989; Walker, S E., Fredrick, K., Recognition and Positioning of mRNA in the Ribosome by tRNAs with Expanded Anticodons, J Mol Biol. 2006 Jul. 14; 360(3): 599-609, doi:10.1016/j.jmb.2006.05.006). Sako et al. 2006 (Sako Y, Usuki F, Suga H. A novel therapeutic approach for genetic diseases by introduction of suppressor tRNA. Nucleic Acids Symp Ser (Oxf). 2006; (50):239-240) describe an approach to read through PTC-containing mRNAs using suppressor tRNA that is introduced to cells by transfection. Nonsense triplet codons and four-base codons were read by the corresponding suppressor tRNAs derived from human tRNA(Ser).

tRNAs with an extended anticodon loop comprising a four-base or five-base anticodon have also been introduced to in vitro translation systems of bacteria to incorporate unnatural amino acids into proteins and address the molecular mechanism of frameshift suppression of two-, three-, four-, five-, and six-base codons with tRNAs containing 6-10 nt in their anticodon loops (US 2006/0177900 A1; WO 2005/007870; Hohsaka T, Ashizuka Y, Murakami H, Sisido M. Five-base codons for incorporation of nonnatural amino acids into proteins. Nucleic Acids Research. 2001; 29(17):3646-3651; Hohsaka T, Sisido M. Incorporation of non-natural amino acids into proteins. Curr Opin Chem Biol. 2002 December; 6(6):809-15; Anderson J C, Magliery T J, Schultz P G. Exploring the limits of codon and anticodon size. Chem Biol. 2002, 9(2):237-44. DOI: 10.1016/S1074-5521(02)00094-7; Hohsaka, T., 2004, Incorporation of Nonnatural Amino Acids into Proteins through Extension of the Genetic Code, Bull. Chem. Soc. Jpn. 77, 1041-1049, DOI: 10.1246/bcsj.77.1041). The yields of incorporation of unnatural amino acids are, however, extremely low, and are influenced by the mRNA context. Further, in approaches to incorporate non-natural (non-standard or non-proteinogenic) amino acids into proteins with the aid of tRNA modified in their anticodon loop, so-called orthogonal translation systems are generally used, i.e. translations systems using engineered aminoacyl tRNA synthetase in order to attach a non-standard amino acid onto the modified tRNA (Wang, K., Sachdeva, A., Cox, D. et al. Optimized orthogonal translation of unnatural amino acids enables spontaneous protein double-labelling and FRET. Nature Chem 6, 393-403 (2014). doi: 10.1038/nchem.1919; Ohtsuki, T., Manabe, T., Sisido, M., Multiple incorporation of non-natural amino acids into a single protein using tRNAs with non-standard structures, 2005, FEBS Lett. 579, 6769-6774, doi: 10.1016/j.febslet.2005.11.010; WO 2005/019415 A2).

There is still a need to counteract the effects of and/or suppressing a frameshift mutation. It is therefore an object of the invention to provide such means, in particular a frameshift mutation suppressor for the treatment of a genetic disease associated with a frameshift mutation, like, for example, neurofibromatosis.

In one aspect the invention provides a synthetic transfer ribonucleic acid (tRNA), the synthetic transfer RNA comprising an extended anticodon loop with a four- or five-base anticodon. The four-base anticodon is configured to base-pair to four, the five-base anticodon is configured to base-pair to five consecutive nucleotide bases on a messenger RNA in order to correct insertions and deletions in consecutive codon base triplets on an mRNA, which change the reading-frame register into a −1 and +1 frame.

The invention provides novel suppressor tRNAs that can be used to suppress a −1 or +1 frameshift mutation with high specificity, in a context-dependent manner, for example, to restore the ability of a cell to synthesize a functional protein from an mRNA having a mutation in its coding sequence, which would otherwise lead to a different protein sequence and a less functional or non-functional protein. The synthetic tRNA of the invention comprises an anticodon loop being extended by one or two nucleotides. The synthetic tRNA of the invention is complementary to four or five bases of two adjacent codons on the mRNA, the first being the codon with an indel (insertion or deletion) and the second being an intact adjacent codon. The synthetic tRNA of the invention with four- or five-base codons base-pairs with the codon and the remaining of the following (in case of a −1 frameshifting) or of the preceding (in case of +1 frameshifting) codon on the mRNA resulting in the incorporation of an amino acid carried by the tRNA into the growing amino acid chain and the correction of the reading frame. In case of a −1 frameshift, unless the synthetic tRNA of the invention is (pre)aminoacylated with a dipeptide, the resulting protein will have one amino acid less than the wild-type protein, i.e. a protein synthesized from the wild-type mRNA, but there is still a good chance that this will lead to a functional protein.

Advantageously, the invention provides synthetic transfer RNA being designed to have an anticodon loop leading to higher binding affinity when compared to prior art suppressor tRNAs used for non-natural amino acid incorporation. The binding to two consecutive codons, the one with the indel (insertion or deletion) and the adjacent unaltered codon, is associated with higher specificity compared to natural two-nucleotide-anticodon suppressor tRNAs. Consequently, the synthetic tRNA of the invention can be designed to effectively bind to a specific mutation site, considerably reducing the risk of unspecific pairing to other partially homologous regions of the mRNA.

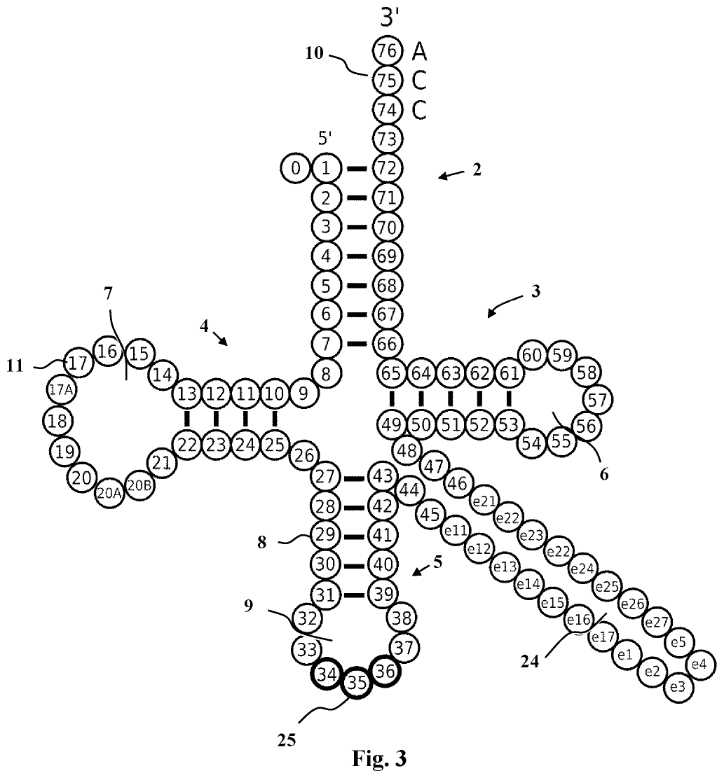

The terms “transfer ribonucleic acid” or “tRNA” refer to RNA molecules with a length of typically 73 to 90 nucleotides, which mediate the translation of a nucleotide sequence in a messenger RNA into the amino acid sequence of a protein. tRNAs are able to covalently bind a specific amino acid at their 3′ CCA tail at the end of the acceptor stem, and to base-pair via a three-nucleotide anticodon in the anticodon loop of the anticodon arm with a three-nucleotide sequence (codon) in the messenger RNA. Some anticodons can pair with more than one codon due to a phenomenon known as wobble base pairing. The secondary “cloverleaf” structure of tRNA comprises the acceptor stem binding the amino acid and three arms (“D arm”, “T arm” and “anticodon arm”) ending in loops (D loop, TψC loop, anticodon loop), i.e. sections with unpaired nucleotides. Aminoacyl tRNA synthetases charge (aminoacylate) tRNAs with a specific amino acid. Each tRNA contains a distinct anticodon triplet sequence that can base-pair to one or more codons for an amino acid. By convention, the nucleotides of tRNAs are often numbered 1 to 76, starting from the 5′-phosphate terminus, based on a “consensus” tRNA molecule consisting of 76 nucleotides, and regardless of the actual number of nucleotides in the tRNA, which are not always of length 76 due to variable portions, such as the D loop in the tRNA (see FIG. 3). Following this convention, nucleotide positions 34-36 of naturally occurring tRNA refer to the three nucleotides of the anticodon, and positions 74-76 refer to the terminating CCA tail. Any “supernumerary” nucleotide can be numbered by adding alphabetic characters to the number of the previous nucleotide being part of the consensus tRNA and numbered according to the convention, for example 20a, 20b etc, or by independently numbering the nucleotides and adding a leading letter, as in case of the variable loop such as e11, e12 etc. (see, for example, Sprinzl M, Horn C, Brown M, Ioudovitch A, Steinberg S. Compilation of tRNA sequences and sequences of tRNA genes. Nucleic Acids Res. 1998; 26(1):148-53). In the following, the tRNA-specific numbering will also be referred to as “tRNA numbering convention” or “transfer RNA numbering convention”.

The terms “synthetic transfer ribonucleic acid” or “synthetic tRNA” refer to a non-naturally occurring tRNA. The term also encompasses analogues to naturally occurring tRNAs, i.e. tRNAs being structurally similar to naturally occurring tRNAs, but being modified in the base component, the sugar component and/or the phosphate component of one or more of the nucleotides, of which the tRNA is composed. The modified tRNA may, for example, have the phosphodiester backbone modified in that the phosphodiester bridge is replaced by a phosphorothioate, phosphoramidate or methyl phosphonate bridge. The sugar component may, for example, be modified at the 2′ OH group, e.g. by dehydroxylating it to a deoxy ribonucleotide, or by replacing it with a methoxy-, methoxyethoxy- or aminoethoxy group. A synthetic transfer ribonucleic acid can, for example, be synthesized chemically and/or enzymatically in vitro, or in a cell based system, e.g. in a bacterial cell in vivo.

The term “codon” refers to a sequence of nucleotide triplets, i.e. three DNA or RNA nucleotides, corresponding to a specific amino acid or stop signal during protein synthesis. A list of codons (on mRNA level) and the encoded amino acids are given in the following:

| Amino | One | |
|---|---|---|
| acid | Letter Code | Codons |
| Ala | A | GCU, GCC, GCA, GCG |
| Arg | R | CGU, CGC, CGA, CGG, AGA, AGG |
| Asn | N | AAU, AAC |
| Asp | D | GAU, GAC |
| Cys | C | UGU, UGC |
| Gln | Q | CAA, CAG |
| Glu | E | GAA, GAG |
| Gly | G | GGU, GGC, GGA, GGG |
| His | H | CAU, CAC |
| Ile | I | AUU, AUC, AUA |
| Leu | L | UUA, UUG, CUU, CUC, CUA, CUG |
| Lys | K | AAA, AAG |
| Met | M | AUG |
| Phe | F | UUU, UUC |
| Pro | P | CCU, CCC, CCA, CCG |
| Ser | S | UCU, UCC, UCA, UCG, AGU, AGC |
| Thr | T | ACU, ACC, ACA, ACG |
| Trp | W | UGG |
| Tyr | Y | UAU, UAC |
| Val | V | GUU, GUC, GUA, GUG |

START: AUG
STOP: UAA, UGA, UAG, abbreviated “X”

The term “sense codon” as used herein refers to a codon coding for an amino acid. The term “stop codon” or “nonsense codon” refers to a codon, i.e. a nucleotide triplet, of the genetic code not coding for one of the 20 amino acids normally found in proteins and signalling the termination of translation of a messenger RNA.

The term “frameshift mutation” refers to an out-of-frame insertion or deletion (collectively called “indels”) of nucleotides with a number not evenly divisible by three. This perturbs the nucleotide sequence decoding which proceeds in steps of three nucleotide bases. The term “−1 frameshift mutation” relates to the deletion of a single nucleotide causing a shift in the reading frame by one nucleotide leading to the first nucleotide of the following codon being read as part of the codon from which the nucleotide has been deleted. Deletion of one nucleotide from the upstream codon along with several triplets (i.e. deletion of 4, 7, 10 etc. nucleotides) is also considered as −1 frameshifting. The term “+1 frameshift mutation” relates to the insertion of a single nucleotide into a triplet, or the deletion of two nucleotides. The result of either event is to shift the reading frame by one nucleotide, such that a nucleotide of an upstream codon is being read as part of a downstream codon. Insertion of one nucleotide along with several triplets (3n+1 nucleotides, n being an integer, i.e. insertion of 4, 7, 10 etc. nucleotides), or deletion of two nucleotides from the upstream codon along with several triplets (i.e. deletion of 5, 8, 11 etc nucleotides) is also considered as +1 frameshifting. Frameshift mutations are implicated in a variety of genetic disorders such as Duchenne muscular dystrophy (DMD), Crohn disease (CD), Tay-Sachs disease (TSD), cystic fibrosis (CF), neuronal ceroid lipofuscinosis (NCL) or neurofibromatosis type 1 (NF1).

The term “anticodon” refers to a sequence of usually three nucleotides that base-pair (non-covalently bind) to the three bases (nucleotides) of the codon on the mRNA. An anticodon may also contain nucleotides with modified bases. The terms “four nucleotide anticodon” or “four base anticodon” relate to an anticodon having four consecutive nucleotides (bases) which pair with four consecutive bases on an mRNA. The terms “quadruplet nucleotide anticodon” or “quadruplet anticodon” may also be used to denote a “four nucleotide anticodon”. The terms “five nucleotide anticodon” or “five base anticodon” relate to an anticodon having five consecutive nucleotides (bases) which bind (base-pair) to five consecutive bases on an mRNA. The terms “quintuplet nucleotide anticodon” or “quintuplet anticodon” may also be used to denote a “five nucleotide anticodon”.

The term “anticodon loop” refers to the unpaired nucleotides of the anticodon arm containing the anticodon. Naturally occurring tRNAs usually have seven nucleotides in their anticodon loop, three of which pair to the codon in the mRNA.

The term “extended anticodon loop” refers to an anticodon loop with a higher number of nucleotides in the loop than in naturally occurring tRNAs. An extended anticodon loop may, for example, contain more than seven nucleotides, e.g. eight, nine, or ten nucleotides. In particular, the term relates to an anticodon loop comprising an anticodon composed of more than three consecutive nucleotides, e.g. four, five or six nucleotides, being able to base-pair with a corresponding number of consecutive nucleotides in an mRNA.

The term “anticodon stem” refers to the paired nucleotides of the anticodon arm that carry the anticodon loop.

The term “T stem” relates to the paired nucleotides of the T arm, which carry the T loop, i.e. the unpaired nucleotides of the T arm.

The term “variable loop” refers to a tRNA loop located between the anticodon arm and the T arm. The number of nucleotides composing the variable loop may largely vary from tRNA to tRNA. The variable loop can thus be rather short or even missing, or rather large, forming, for example, a helix. The term “variable arm” may synonymously be used for the term “variable loop”.

The terms “codon base triplet” or “anticodon base triplet”, when used herein, refer to sequences of three consecutive nucleotides which form a codon or anticodon. Synonymously, the terms “three nucleotide codon” (also “three base codon”) or “three nucleotide anticodon” (also “three base anticodon”), or abbreviations thereof, e.g. “3nt codon” or “3nt anticodon”, may be used.

The term “base pair” refers to a pair of bases, or the formation of such a pair of bases, joined by hydrogen bonds. One of the bases of the base pair is usually a purine, and the other base is usually a pyrimidine. In RNA the bases adenine and uracil can form a base pair and the bases guanine and cytosine can form a base pair. However, the formation of other base pairs (“wobble base pairs”) is also possible, e.g. base pairs of guanine-uracil (G-U), hypoxanthine-uracil (I-U), hypoxanthine-adenine (I-A), and hypoxanthine-cytosine (I-C). The term “being able to base-pair” refers to the ability of nucleotides or sequences of nucleotides to form hydrogen-bond-stabilised structures with a corresponding nucleotide or nucleotide sequence.

The term “base”, as used herein, for example in terms like “the bases A, C, G or U”, encompasses or is synonymously used to the term “nucleotide”, unless the context clearly indicates otherwise.

“PTC” refers to a premature termination codon. This is a stop codon introduced into a coding nucleic acid sequence by a nonsense mutation, i.e. a mutation in which a sense codon coding for one of the twenty proteinogenic amino acids specified by the standard genetic code is changed to a chain-terminating codon. The term thus refers to a premature stop signal in the translation of the genetic code contained in mRNA.

The terms “Crohn disease” or “Crohn's disease” relate to a gastro-intestinal inflammatory disease associated with the NOD2 gene. A common mutation associated with this disease is an insertion of cytosine at position 3020.

The term “Tay-Sachs disease” relates to a genetic disorder resulting in nerve cells destruction and is apparent at early childhood at around 2-3 months after birth. It leads to severe movement disability, hearing loss, seizures etc. Death often occurs in early childhood.

The term “Duchenne muscular dystrophy” (DMD) (also “Becker muscular dystrophy”, BMD) refers to an X-linked recessive genetic disorder characterized by progressive muscle degeneration and weakness caused by an absence of a functional dystrophin protein. The absence of dystrophin can be caused by a nonsense mutation in the dystrophin gene.

“Neurofibromatosis type 1” (NF1 or NF-1), also called “Recklinghausen disease”, is an autosomal dominant inherited disorder caused by the mutation of the NF1 gene on chromosome 17 coding for neurofibromin. NF1 causes tumours along the nervous system.

The term “cystic fibrosis” refers to a genetic disorder inherited in an autosomal recessive manner. It is caused by mutations in both copies of the gene for the cystic fibrosis transmembrane conductance regulator (CFTR) protein. Cystic fibrosis can, for example, be caused by a frameshift mutation resulting in the introduction of a premature stop codon (see, e.g., Iannuzzi M C, Stern R C, Collins F S, Hon C T, Hidaka N, Strong T, Becker L, Drumm M L, White M B, Gerrard B, Dean, M., 1991, Two frameshift mutations in the cystic fibrosis gene, Am J Hum Genet. 48(2):227-31, PMID: 1990834; PMCID: PMC1683026).

The term “Neuronal ceroid lipofuscinosis” refers to neurodegenerative lysosomal storage disease linked to alterations in tripeptides in lysosomes. Neuronal ceroid lipofuscinosis, for example among other mutations, can result from a frameshift mutation in the tripeptidyl peptidase I (TPP1) or CLN2-gene (see, for example, Gardner E, Bailey M, Schulz A, Aristorena M, Miller N, Mole S E. Mutation update: Review of TPP1 gene variants associated with neuronal ceroid lipofuscinosis CLN2 disease. Hum Mutat. 2019; 40(11):1924-1938. doi:10.1002/humu.23860).

The term “frameshift suppression” refers to mechanisms masking the effects of a frameshift mutation and at least partly restoring the wild-type phenotype.

The term “suppressor tRNA” relates to a tRNA altering the reading of a messenger RNA in some translation systems. An example of a suppressor tRNA is a tRNA carrying an amino acid and being able to base-pair to mutated codons covering two consecutive codons, of which one is intact and one has an insertion or deletion. The translation system can thus correct the reading frame.

The term “homology” in relation to a nucleic acid refers to the degree of similarity or identity between the nucleotide sequence of the nucleic acid and the nucleotide sequence of another nucleic acid. Homology is determined by comparing a position in the first sequence with a corresponding position in the second sequence in order to determine whether identical nucleotides are present at that position. It may be necessary to take sequence gaps into account in order to produce the best possible alignment. For determining the degree of similarity or identity between two nucleic acids it is preferable to take a minimum length of the nucleic acids to be compared into account, for example at least 25%, 30%, 40%, 50%, 60%, 70%, 80%, 90%, 91%, 92%, 93%, 94%, 95%, 96%, 97%, 98%, 99%, 99.2% or 99.5% of the nucleotides in the respective sequences. Preferably the full length of the respective nucleic acid(s) is used for comparison. The degree of similarity or identity of two sequences can be determined by using a computer program such as muscle (Edgar, R. C. (2004), Muscle: multiple sequence alignment with high accuracy and high throughput, Nucleic Acids Res., 32, 1792-1797, doi: 10.1093/nar/gkh340) or mafft (Katoh, K. and Standley, D. M. (2013) MAFFT Multiple Sequence Alignment Software Version 7, Mol. Biol. Evol., 30, 772-780, doi.org/10.1093/molbev/mst010). Where such terms like “x % homologous to” or “homology of x %” are used herein, this means that two nucleic acids have a sequence identity or similarity, preferably sequence identity, of x %, e.g. 50%.

The term “aminoacylation” relates to the enzymatic reaction in which a tRNA is charged with an amino acid. An aminoacyl tRNA synthetase (aaRS) catalyses the esterification of a specific cognate amino acid or its precursor to a compatible cognate tRNA to form an aminoacyl-tRNA. The term “aminoacyl-tRNA” thus relates to a tRNA with an amino acid attached to it. Each aminoacyl-tRNA synthetase is highly specific for a given amino acid, and, although more than one tRNA may be present for the same amino acid, there is only one aminoacyl tRNA synthetase for each of the 20 proteinogenic amino acids. The terms “charge” or “load” may also be used synonymously for “aminoacylate”. The term “aminoacylated” in relation to the synthetic tRNA of the invention relates to a synthetic tRNA already charged (precharged) with an amino acid or a dipeptide, such that the tRNA is already acylated when entering the target cell. The term “preaminoacylated” may synonymously be used in this context.

The term “modified nucleotides” (or “unusual nucleotides”) in reference to tRNA relates to nucleotides having modified or unusual nucleotide bases, i.e. other than the usual bases adenine (A), uracil (U), guanine (G) and cytosine (C). Examples of modified nucleotides include 4-acetylcytidine (ac4c), 5-(carboxyhydroxymethyl)uridine (chm5u), 2′-O-methylcytidine (cm), 5-carboxymethylaminomethyl-2-thiouridine (cmnm5s2u), 5-carboxymethylaminomethyluridine (cmnm5u), dihydrouridine (d), 2′-O-methylpseudouridine (fm), beta, D-galactosylqueuosine (gal q), 2′-O-methylguanosine (gm), inosine (i), N6-isopentenyladenosine (i6a), 1-methyladenosine (m1a), 1-methylpseudouridine (m1f), 1-methylguanosine (m1g), 1-methylinosine (m1i), 2,2-dimethylguanosine (m22g), 2′-O-methyladenosine (am), 2-methyladenosine (m2a), 2-methylguanosine (m2g), 3-methylcytidine (m3c), 5-methylcytidine (m5c), N6-methyladenosine (m6a), 7-methylguanosine (m7g), 5-methylaminomethyluridine (mam5u), 5-methoxyaminomethyl-2-thiouridine (mam5s2u), beta, D-mannosylqueuosine (man q), 5-methoxycarbonylmethyl-2-thiouridine (mcm5s2u), 5-methoxycarbonylmethyluridine (mcm5u), 5-carbamoylmethyluridine (ncm5U), 5-carbamoylmethyl-2′-O-methyluridine (ncm5Um), 5-methoxyuridine (mo5u), 2-methylthio-N6-isopentenyladenosine (ms2i6a), N-((9-beta-D-ribofuranosyl-2-methylthiopurine-6-yl)carbamoyl)threonine (ms2t6a), N-((9-beta-D-ribofuranosylpurine-6-yl)N-methylcarbamoyl)threonine (mt6a), uridine-5-oxyacetic acid-methylester (mv), uridine-5-oxyacetic acid (o5u), wybutoxosine (osyw), pseudouridine (p, Ψ), queuosine (q), 2-thiocytidine (s2c), 5-methyl-2-thiouridine (s2t), 2-thiouridine (s2u), 4-thiouridine (s4u), 5-methyluridine (t), N-((9-beta-D-ribofuranosylpurine-6-yl)-carbamoyl)threonine (t6a), 2′-O-methyl-5-methyluridine (tm), 2′-O-methyluridine (um), wybutosine (yw), 3-(3-amino-3-carboxy-propyl)uridine, (acp3)u (x),

The term “corresponding modified nucleotide” relates to a modified nucleotide at a given position in a sequence, which base has been modified based on the usual, i.e. unmodified, base of the nucleotide at the same position in the original sequence to be compared with the sequence containing the modified nucleotide. A corresponding modified nucleotide is thus any nucleotide that, in a cell, is usually produced from a usual nucleotide by modifying the usual nucleotide. A modified nucleotide corresponding to uridine, for example, is thus any nucleotide derived by the modification of uridine. As an example, 5-(carboxyhydroxymethyl)uridine (chm5u) at a particular position in a sequence may be a modified nucleotide corresponding to uridine at the same position in the original sequence. Further modified nucleotides corresponding to uridine are, for example, 5-methyluridine (t), 2′-O-methyl-5-methyluridine (tm), 2′-O-methyluridine (um), or 5-methoxyuridine (mo5u). Inosine, as another example, is produced from adenosine and thus is a modified nucleotide corresponding to adenosine.

The synthetic transfer RNA of the invention may be synthesized based on a naturally occurring tRNA. However, the tRNA of the invention is preferably designed computationally (“in silico”) and synthesized in vitro chemically and/or enzymatically. The computational design of a synthetic tRNA according to the invention allows the design and synthesis of a tRNA that does not interfere with other tRNAs present in the cell. The synthetic tRNA of the invention is selected or designed in such a manner that an aminoacyl tRNA synthetase that naturally occurs in a living cell, preferably a mammalian cell, e.g. a human cell, is able to charge the tRNA with a specific amino acid that is encoded by the codon adjacent to the mutated codon or by the wild-type codon receiving indels.

In order to counteract a possible destabilization due to the anticodon loop expansion, the synthetic tRNA of the invention may be further structurally modified, within or outside its anticodon loop, to have a high stability. Preferably, the tRNA of the invention is designed to have at least the same or a higher stability than a naturally occurring tRNA in which the natural three-nucleotide anticodon is replaced with a four- or five-nucleotide anticodon, or to have at least the same or a higher stability compared to a naturally occurring tRNA for the same cognate amino acid. The term “stability” relates to the adoption of a stable and correctly folded and functional conformation of the tRNA in the absence of a translation factor, and can be predicted by estimating the free energy change upon folding, and/or to a stable binding to an elongation factor leading to an interaction of the tRNA with the elongation factor allowing elongation. “Higher stability” means, for example, that the favourable free energy change upon the tRNA folding to a functional state is greater. This includes a higher fraction of tRNA molecules in the desired configuration, i.e. adequately or completely folded. “Higher stability” can also mean a higher stability of binding to an elongation factor, for example, a higher binding affinity, still allowing, however, a preferably smooth elongation of translation. The stability of a synthetic transfer RNA of the invention with a four-nucleotide anticodon is compared with a naturally occurring transfer RNA whose three-nucleotide anticodon has been replaced with a four-nucleotide anticodon, or with a naturally occurring tRNA for the same cognate amino acid, and the stability of a synthetic transfer RNA of the invention with a five-nucleotide anticodon is compared with a naturally occurring transfer RNA whose three-nucleotide anticodon has been replaced with a five-nucleotide anticodon, or with a naturally occurring tRNA for the same cognate amino acid. A high stability of the synthetic tRNA of the invention leads to an increased concentration of the folded tRNA within the cell compared to less stable tRNA. Further preferred, the synthetic tRNA of the invention is configured to have less stability in a complex with the elongation factor (e.g. eEF1A), thus increasing the functional promiscuity of the tRNA in translation and consequently the suppression activity.

The tRNA may, for example, be modified regarding the nucleotide composition of its anticodon arm or components outside the anticodon arm, for example of its D-arm, T-arm or variable arm. It is preferred, for example, that the synthetic tRNA of the invention has a C, i.e. the nucleotide C, at position 32 and/or an A, i.e. the nucleotide A, at position 37 in the anticodon loop, the numbering following the above-mentioned tRNA numbering convention. It is further preferred that the anticodon loop is flanked by a G-C or C-G pair, i.e. to have a G-C or C-G pair at the end of the anticodon stem in direction of the anticodon loop. The terms “G-C pair” or “C-G pair” relate to the bases (nucleotides) C and G paired via hydrogen bonds and being separated, in 5′-3′ direction, by the anticodon loop. According to tRNA numbering convention, the G-C or C-G pair would take the positions 31 and 39, i.e., in case of a “G-C” pair, G at position 31 and C at position 39, or, in case of a “C-G” pair, C at position 31 and G at position 39. In an alternative preferred embodiment, a U-A pair (in 5′-3′ direction) is flanking the anticodon loop, i.e. is located at the end of the anticodon stem in direction of the anticodon loop, i.e. at positions 31 and 39 according to tRNA numbering convention.

Examples of anticodon arms with anticodon loops suitable for use as a suppressor of a +1 frameshift mutation or a −1 frameshift mutation and having U at position 31 and A at position 39 (U31-A39 anticodon arm) are the following:

+1 frameshift:
(SEQ ID NO: 17)
AUGGUCUNNNNAAACCAU
−1 frameshift:
(SEQ ID NO: 18)
AUGGUCUNNNNNAAACCAU

The underlined N's represent a 4nt or 5nt anticodon.

In a preferred embodiment of the invention, the synthetic tRNA of the invention comprises a T stem having the following structure:

N₁CGGG-T-Loop-CCCGN₂,

wherein

N₁=A, G, or C

N₂=C, U, or G

The first five nucleotides (N₁CGGG) and the last five nucleotides (CCCGN₂) pair to form the T stem, with the T loop in-between.

Preferred combinations of N₁ and N₂ are:

N₁=G, N₂=C

N₁=A, N₂=U

N₁=G, N₂=U

N₁=C, N₂=G

In a preferred embodiment the T arm has the following general sequence (SEQ ID NO: 19):

N₁CGGGNNNNNNNCCCGN₂,

N₁ and N₂ being as defined above, and

the inner consecutive Ns forming the T loop, N being any nucleotide or any corresponding modified nucleotide.

The T loop may have the following sequence:

UUCGAAU.

In a preferred embodiment, the T arm may thus have the following sequence SEQ ID NO: 20:

N₁CGGGUUCGAAUCCCGN₂,

wherein N₁ and N₂ are as defined above.

Especially preferred embodiments of the T arm are:

GCGGG-T-loop-CCCGC,
ACGGG-T-loop-CCCGU,
GCGGG-T-loop-CCCGU, or
CCGGG-T-loop-CCCGG.

The T loop preferably has the sequence given above (UUCGAAU), such that the T arm preferably has the following sequences:

(SEQ ID NO: 21)
GCGGGUUCGAAUCCCGC,
(SEQ ID NO: 22)
ACGGGUUCGAAUCCCGU,
(SEQ ID NO: 23)
GCGGGUUCGAAUCCCGU,
or
(SEQ ID NO: 24)
CCGGGUUCGAAUCCCGG.

It has been shown that a synthetic tRNA of the invention comprising a T arm as given above has excellent stability and is useful for use as suppressor tRNA, notably not only for suppressing frameshift mutations but also for suppressing nonsense mutations (mutations converting a sense codon into a stop codon, PTC).

In a preferred embodiment of the invention the synthetic tRNA comprises a variable loop having the following sequence (SEQ ID NO: 25):

UGGGGNNNNNNCCCCGC

An example of a preferred embodiment of the variable loop is presented in the sequence below (SEQ ID NO: 26):

UGGGGUCCACUCCCCGC

As with the tRNA comprising the T arm described above, a synthetic tRNA comprising the above variable loop, has been shown to have excellent stability and can advantageously be used for suppressing frameshift mutations, but also for suppressing nonsense mutations, for example.

A synthetic tRNA of the invention may comprise the T arm or the variable loop described above each alone, or in combination. It is further preferred for a tRNA of the invention to include a U31-A39 anticodon arm, i.e. an anticodon arm having U at position 31 and A at position 39. The anticodon arm may have the sequence given in SEQ ID NO: 17 or SEQ ID NO: 18.

The skilled person is aware of the fact that a tRNA is aminoacylated with a specific amino acid by a specific aminoacyl tRNA synthetase (aaRS), and that the aaRS is able to recognize its cognate tRNA through unique identity elements at the acceptor stem and/or anticodon loop of the tRNA. In order to provide a tRNA which is loaded with its cognate amino acid in vivo, the skilled person will design the synthetic tRNA of the invention with suitable unique identity elements. It is also possible to aminoacylate a synthetic tRNA of the invention with a flexizyme and transfect it into an eukaryotic cell as aminoacyl-tRNA (Goto, Y., Kato, T. and Suga, H., Flexizymes for genetic code reprogramming, Nat Protoc., 6(6), 779, 2011, doi:10.1038/nprot.2011.331).

The tRNA of the invention preferably has a low sequence identity to any naturally occurring tRNA, and has preferably a sequence identity of less than 70%, 65%, 60%, 65% or 50%, especially preferred of less than 49%, 48%, 47%, 46%, 45%, 44% or 43%, preferably comparing the full length of the tRNA.

In the synthetic transfer RNA according to the invention the anticodon loop has been extended by a large enough number of nucleotides to accommodate the four or five nucleotide anticodon and to allow base-pairing with an mRNA. The anticodon loop of a synthetic transfer RNA of the invention may, for example, consist of 7-12, preferably 7 to 10 or 8-10, further especially preferred 8 or 9 nucleotides.

The extended anticodon loop of the synthetic tRNA of the invention comprises, in one embodiment, a four-base anticodon, which is able to base-pair to a four-base codon, i.e. a codon base triplet on a targeted mRNA with an additional nucleotide. In another embodiment, the synthetic tRNA of the invention comprises a five-base anticodon, including a base doublet being able to base-pair to a codon doublet left from a codon triplet on the mRNA after deletion of a nucleotide in that codon, whereas the neighbouring anticodon base triplet preferably base-pairs to a sense codon preceding or following, i.e. 5′ or 3′ to the codon with the deletion on the mRNA. The terms “preceding” or “following” relate to the direction of translation, i.e. the 5′-3′ direction of the mRNA.

An example of a five-nucleotide anticodon in the extended anticodon loop of the synthetic tRNA of the invention is GAUUC (in 5′-3′ direction, or CUUAG in 3′-5′ direction), matching with GAAAUC (5′-3′) in the unmutated mRNA, where UC is able to base-pair with the base doublet GA left after deletion of an A in the codon GAA, and GAU is able to base-pair with the codon AUC coding for isoleucine.

The synthetic transfer RNA according to the invention may be aminoacylated, i.e. carrying an amino acid or a dipeptide at the end of its acceptor stem. Preferably, the tRNA is aminoacylated with an amino acid being encoded by a sense codon base-pairing with the four or five nucleotide anticodon. The synthetic tRNA of the invention can be chemically and/or enzymatically aminoacylated with a single amino acid or dipeptide. The loading of a tRNA with a dipeptide can be accomplished with methods known to those skilled in the art (see, for example, Maini R, Dedkova L M, Paul R, Madathil M M, Chowdhury S R, Chen S, Hecht S M, 2015, Ribosome-Mediated Incorporation of Dipeptides and Dipeptide Analogues into Proteins in Vitro, J. Am. Chem. Soc., 137, 11206-11209, doi 10.1021/jacs.5b03135). Engineered bacterial tRNA synthetases or RNA-based catalysts may, for example, be used to aminoacylate the tRNA with a dipeptide. A dipeptide is preferably composed of the amino acids encoded by the consecutive unmutated codons in case of a deletion. The use of a synthetic tRNA aminoacylated with such a dipeptide would not only result in the intended suppression of a −1 frameshift and the production of a non-truncated protein, but also in the production of a protein having the amino acid sequence of the wild-type protein.

In preferred embodiments, the synthetic transfer RNA of the invention has or comprises a) a sequence being composed of, in 5′ to 3′ direction, consecutive sequence parts A, B and C, part A having or comprising one of the sequences according to SEQ ID NO: 01, SEQ ID NO: 02, SEQ ID NO: 07, SEQ ID NO: 08, SEQ ID NO: 27, SEQ ID NO: 28, SEQ ID NO: 29 and SEQ ID NO: 30, part B having or comprising one of the sequences according to SEQ ID NO: 03, SEQ ID NO: 04, SEQ ID NO: 06, SEQ ID NO 25, SEQ ID NO: 31 and SEQ ID NO: 32, and part C having or comprising one of the sequences according to SEQ ID NO: 5, SEQ ID NO: 33 and SEQ ID NO: 34, or b) a sequence having at least 90%, preferably at least 95%, 96%, 97%, 98% or 99% sequence identity with one of the sequences according to a) above, or c) a sequence according to one of sequences of a) or b) above, where at least one of the nucleotides is replaced with a corresponding modified nucleotide. The tRNA of the invention may be composed of any combination of the parts A, B and C above, provided the order of parts, in 5′-3′ direction, is A-B-C.

In a preferred embodiment the synthetic tRNA of the invention is composed of three sequence parts A, B and C, which are covalently bonded in the 5′ to 3′ direction, e.g. via phosphodiester bonds.

In 4-nt-anticodon tRNAs the part A comprises the 4-nt-anticodon (underlined “N”s below) and preferably has the sequence of SEQ ID NO: 1, SEQ ID NO: 2, SEQ ID NO: 27 or SEQ ID NO: 28.

SEQ ID NO: 1
NNCAGNNUGNNCGAGNNGUCUAAGNNNNNNGCCUNNNNAUGCNNNUN
SEQ ID NO: 2
NNCAGNNUGNNCGAGNNGUCUAAGNNNNNNGCCUNNNNAGCNNNUN
SEQ ID NO: 27
NNCAGNNUGNNCGAGNNGUCUAAGNNNNNNGUCUNNNNAAACNNNUN
SEQ ID NO: 28
NNCAGNNUGNNCGAGNNGUCUAAGNNNAUGGUCUNNNNAAACCAUUN

N=any nucleotide or any corresponding modified nucleotide.

It is to be noted here that the possible combinations of the unknown nucleotides (N) in a sequence of a tRNA of the invention, i.e. the positions in the sequence that can be any nucleotide or corresponding modified nucleotide, except those that are not involved in a base pair like those in one of the loops, e.g. the T loop or the anticodon loop, are limited to those being able to form a functional tRNA molecule via base pairing.

Part B may be of variable length and comprises or consists of the variable loop, and preferably has the sequences of SEQ ID NO: 3, 4, 6, 25, 31 or 32 (N=any nucleotide, or any corresponding modified nucleotide).

SEQ ID NO: 3
NNNNNNNNNNN
SEQ ID NO: 4
NNNNNNNNNNNNNNN
SEQ ID NO: 6
UGGGGUCACUCCCCG
SEQ ID NO: 25
UGGGGNNNNNNCCCCGC
SEQ ID NO: 31
NNNNNNNNNNNNNNNN
SEQ ID NO: 32
UGGGGUCCACUCCCCGC

Part C preferably has the sequence according to SEQ ID NO: 5, 33 or 34

SEQ ID NO: 5
CNUGGNUUCGAAUNCCANNNCUGNNACCA
SEQ ID NO: 33
CNCGGGNNNNNNNCCCGNNNCUGNNACCA
SEQ ID NO: 34
CNCGGGUUCGAAUCCCGNNNCUGNNACCA

Examples of preferred 4-nt-anticodon tRNA of the invention composed of parts A to C above have the following sequences (SEQ ID NO: 9-12, 35-38; N=any nucleotide, anticodon underlined):

SEQ ID NO: 9
NNCAGNNUGNNCGAGNNGUCUAAGNNNNNNGCCUNNNNAUGCNNNUNNNNN
NNNNNNNCNUGGNUUCGAAUNCCANNNCUGNNACCA
SEQ ID NO: 10
NNCAGNNUGNNCGAGNNGUCUAAGNNNNNNGCCUNNNNAGCNNNUNNNNNN
NNNNNNCNUGGNUUCGAAUNCCANNNCUGNNACCA
SEQ ID NO: 11
NNCAGNNUGNNCGAGNNGUCUAAGNNNNNNGCCUNNNNAUGCNNNUNUGGG
GUCACUCCCCGCNUGGNUUCGAAUNCCANNNCUGNNACCA
SEQ ID NO: 12
NNCAGNNUGNNCGAGNNGUCUAAGNNNNNNGCCUNNNNAGCNNNUNUGGGG
UCACUCCCCGCNUGGNUUCGAAUNCCANNNCUGNNACCA
SEQ ID NO: 35
NNCAGNNUGNNCGAGNNGUCUAAGNNNNNNGUCUNNNNAAACNNNUNNNNN
NNNNNNNNNNNNNNCNCGGGNNNNNNNCCCGNNNCUGNNACCA
SEQ ID NO: 36
NNCAGNNUGNNCGAGNNGUCUAAGNNNNNNGUCUNNNNAAACNNNUNUGGG
GUCCACUCCCCCGCCNCGGGNNNNNNNCCCGNNNCUGNNACCA
SEQ ID NO: 37
NNCAGNNUGNNCGAGNNGUCUAAGNNNNNNGUCUNNNNAAACNNNUNUGGG
GUCCACUCCCCCGCCNCGGGUUCGAAUCCCGNNNCUGNNACCA
SEQ ID NO: 38
NNCAGNNUGNNCGAGNNGUCUAAGNNNAUGGUCUNNNNAAACNNNUNUGGG
GUCCACUCCCCCGCCNCGGGUUCGAAUCCCGNNNCUGNNACCA

In the case of a 5-nt-anticodon, tRNAs part A comprises the 5-nt-anticodon (underlined “N”s below) and preferably has the sequence of SEQ ID NO: 7, SEQ ID NO: 8, SEQ ID NO: 29 or SEQ ID NO: 30 (N=any nucleotide).

Part A:

SEQ ID NO: 7
NNCAGNNUGNNCGAGNNGUCUAAGNNNNNNGCCUNNNNNAUGCNNNUN
SEQ ID NO: 8
NNCAGNNUGNNCGAGNNGUCUAAGNNNNNNGCCUNNNNNAGCNNNUN
SEQ ID NO: 29
NNCAGNNUGNNCGAGNNGUCUAAGNNNNNNGUCUNNNNNAAACNNNUN
SEQ ID NO: 30
NNCAGNNUGNNCGAGNNGUCUAAGNNNAUGGUCUNNNNNAAACCAUUN

Part B may be of variable length and comprises or consists of the variable loop. In preferred embodiments, part B has one of the following sequences:

SEQ ID NO: 3
NNNNNNNNNNN
SEQ ID NO: 4
NNNNNNNNNNNNNNN
SEQ ID NO: 6
UGGGGUCACUCCCCG
SEQ ID NO: 25
UGGGGNNNNNNCCCCGC
SEQ ID NO: 31
NNNNNNNNNNNNNNNNN
SEQ ID NO: 32
UGGGGUCCACUCCCCGC

Part C preferably has the following sequence:

SEQ ID NO: 5
CNUGGNUUCGAAUNCCANNNCUGNNACCA
SEQ ID NO: 33
CNCGGGNNNNNNNCCCGNNNCUGNNACCA
SEQ ID NO: 34
CNCGGGUUCGAAUCCCGNNNCUGNNACCA

A 5-nt-anticodon tRNA composed of parts A to C of the invention preferably has the following sequences (N=any nucleotide; anticodon underlined):

SEQ ID NO: 13
NNCAGNNUGNNCGAGNNGUCUAAGNNNNNNGCCUNNNNNAUGCNNNUNNNN
NNNNNNNNCNUGGNUUCGAAUNCCANNNCUGNNACCA
SEQ ID NO: 14
NNCAGNNUGNNCGAGNNGUCUAAGNNNNNNGCCUNNNNNAGCNNNUNNNNN
NNNNNNNCNUGGNUUCGAAUNCCANNNCUGNNACCA
SEQ ID NO: 15
NNCAGNNUGNNCGAGNNGUCUAAGNNNNNNGCCUNNNNNAUGCNNNUNUGG
GGUCACUCCCCGCNUGGNUUCGAAUNCCANNNCUGNNACCA
SEQ ID NO: 16
NNCAGNNUGNNCGAGNNGUCUAAGNNNNNNGCCUNNNNNAGCNNNUNUGGG
GUCACUCCCCGCNUGGNUUCGAAUNCCANNNCUGNNACCA
SEQ ID NO: 39
NNCAGNNUGNNCGAGNNGUCUAAGNNNNNNGUCUNNNNNAAACNNNUNNNN
NNNNNNNNNNNNNNNCNCGGGNNNNNNNCCCGNNNCUGNNACCA
SEQ ID NO: 40
NNCAGNNUGNNCGAGNNGUCUAAGNNNNNNGUCUNNNNNAAACNNNUNUGG
GGUCCACUCCCCCGCCNCGGGNNNNNNNCCCGNNNCUGNNACCA
SEQ ID NO: 41
NNCAGNNUGNNCGAGNNGUCUAAGNNNNNNGUCUNNNNNAAACNNNUNUGG
GGUCCACUCCCCCGCCNCGGGUUCGAAUCCCGNNNCUGNNACCA
SEQ ID NO: 42
NNCAGNNUGNNCGAGNNGUCUAAGNNNAUGGUCUNNNNNAAACNNNUNUGG
GGUCCACUCCCCCGCCNCGGGUUCGAAUCCCGNNNCUGNNACCA

In the above sequences, N stands for any of the bases A, C, G or U, or any modified base. Preferably, the base doesn't violate the base pairing as given in FIG. 3. Allowed base pairs are, for example, G-C, C-G, A-U, U-A, and wobble base pairs like G-U, U-G, I-U, U-I, I-A, A-I and I-C, C-I.

The symbols G, C, A or U, as used herein, may represent the unmodified or any corresponding modified base (see below). The tRNA of the invention may thus contain one or more modified nucleotides.

For clarification, it is noted that the synthetic transfer RNA of the invention may or may not be synthesized to contain any modified nucleotides. The synthetic transfer RNA of the invention may thus not contain any modified nucleotide. However, after entering a cell, one or more nucleotides of that synthetic tRNA may nevertheless be modified within the cell by the cellular enzymatic machinery. Consequently, a synthetic tRNA of the invention, which has been designed, synthesized and administered without any modified nucleotide, may, in a living cell, contain one or more modified nucleotides due to modifications the cell has made to them. In fact, it is preferred that the synthetic tRNA of the invention is synthesized and also administered without containing any modified nucleotides and to leave any modifications to the cell.

If a synthetic tRNA of the invention is synthesized with modified nucleotides, such that the tRNA already contains modified nucleotides prior to administration, it is preferred that the tRNA of the invention contains one or more of the following modified nucleotides (Table 1):

TABLE 1
Possible modified nucleotides and positions within the tRNA
(position numbering according to the specific tRNA numbering
convention for a generalized “consensus” tRNA, see also FIG. 3)

| Position | Modification |
|---|---|
| 1 | Ψ |
| 4 | cm, am |
| 9 | m1g |
| 12 | ac4c |
| 16 | d |
| 17 | d |
| 18 | m2g |
| 20, 20a-b | d |
| 26 | m22g |
| 28 | Ψ |
| 29 | Ψ |
| 30 | Ψ |
| 32 | Ψ, 2′O-methylribose, cm |
| 34 | I, Ψ, m5c, cm, gm, 2′O-methylribose, q, mcm5u, |
| | ncm5u, ncm5um, mcm5s2u, |
| 35 | Ψ |

m1g, 1-methylguanosine; am, 2′-O-methyladenosine; cm, 2′-O-methylcytidine; gm, 2′-O-methylguanosine; Ψ, pseudouridine; m2g, N2-methylguanosine; ac4c, N4-acetylcytidine; d, dihydrouridine; m22g, N2,N2-dimethylguanosine; m2g, N2-methylguanosine; I, inosine; m5c, 5-methylcytidine; mcm5u, 5-methoxycarbonylmethyluridine; mcm5s2u, 5-methoxycarbonyl-methyl-2-thiouridine; ncm5u, 5-carbamoylmethyluridine; ncm5 um, 5-carbamoylmethyl-2′-O-methyluridine; q, queuosine; m5c, 5-methylcytidine.

As mentioned above, an unmodified nucleotide in a sequence for a synthetic tRNA of the invention may be replaced with a corresponding modified nucleotide. The symbols A, C, G or U in the above sequences for tRNAs of the invention may therefore represent an unmodified or any corresponding modified base. An A in a sequence may, for example, represent an adenine nucleotide (A) or a corresponding modified nucleotide, e.g. 1-methyladenosine (m1a). During synthesis of the tRNAs, the bases used can be unmodified bases. The bases of the synthesized tRNA may, however, be modified chemically and/or enzymatically in vitro. Once introduced or incorporated in a cell, the tRNAs, whether in vitro synthesized with unmodified or modified nucleotides, may be modified by the cell.

In a further aspect the invention relates to the synthetic transfer RNA according to the first aspect of the invention for use as a medicament. The transfer RNA of the invention is especially useful for treating patients with a disease associated with a frameshifting causing the absence of a functional protein or the dysfunction of a protein. Examples for diseases, in which the tRNA of the invention may advantageously be employed are neurofibromatosis type 1, Duchenne muscular dystrophy, Crohn disease, cystic fibrosis, neuronal ceroid lipofuscinosis and Tay-Sachs disease. Suitable compositions or means for delivering tRNAs to a cell are known. These include viral vectors such as adeno-associated virus (AAV)-based viral vectors, encapsulation in or coupling to nanoparticles.

In a further aspect the invention relates to a method of treating a person having a disease associated with a frameshift mutation, comprising administering an effective amount of the synthetic transfer RNA of the invention or of a composition comprising the synthetic transfer RNA of the invention to the person. In a preferred embodiment the method is for treating neurofibromatosis type 1, Duchenne muscular dystrophy, Crohn disease, cystic fibrosis, neuronal ceroid lipofuscinosis and/or Tay-Sachs disease.

In a still further aspect the invention relates to a synthetic transfer RNA having an extended anticodon loop and comprising:

a) a variable arm having or comprising the sequence of SEQ ID NO: 32, and/or

b) a T arm having or comprising one of the sequences according to SEQ ID NO: 19, SEQ ID NO: 20, SEQ ID NO: 21, SEQ ID NO: 22, SEQ ID NO: 23 and SEQ ID NO: 24, and or

c) U at position 31 and A at position 39 according to tRNA numbering convention.

The tRNA of this aspect of the invention, in particular a tRNA comprising a variable arm having or comprising the sequence of SEQ ID NO: 32, or a T arm having or comprising one of the sequences according to SEQ ID NO: 19, SEQ ID NO: 20, SEQ ID NO: 21, SEQ ID NO: 22, SEQ ID NO: 23 and SEQ ID NO: 24, or comprising a combination of the variable arm having or comprising the sequence of SEQ ID NO: 32 and a T arm having or comprising one of the sequences according to SEQ ID NO: 19, SEQ ID NO: 20, SEQ ID NO: 21, SEQ ID NO: 22, SEQ ID NO: 23 and SEQ ID NO: 24, is stable in a cellular environment, e.g. within a mammalian cell, and useful for suppressing a nonsense or frameshift mutation. The extended anticodon loop may comprise a 4nt-, 5nt- or 6nt-anticodon for suppressing a frameshift or nonsense mutation. The tRNA can thus advantageously used as a suppressor molecule and for treating persons having a disease associated with such a mutation, for example neurofibromatosis type 1, Duchenne muscular dystrophy, Crohn disease, cystic fibrosis, Neuronal ceroid lipofuscinosis or Tay-Sachs disease.

The tRNA of this aspect of the invention preferably has an anticodon arm having U at position 31 and A at position 39. The anticodon arm may, for example, have the sequence of SEQ ID NO: 17 or SEQ ID NO: 18. The tRNA of the aspect of the invention may comprise such an anticodon arm alone, in combination with the variable loop described above, in combination with the T arm described above or in combination with the variable loop and the T arm described above.

The invention will be described in the following by way of examples and the appended figures for illustrative purposes only.

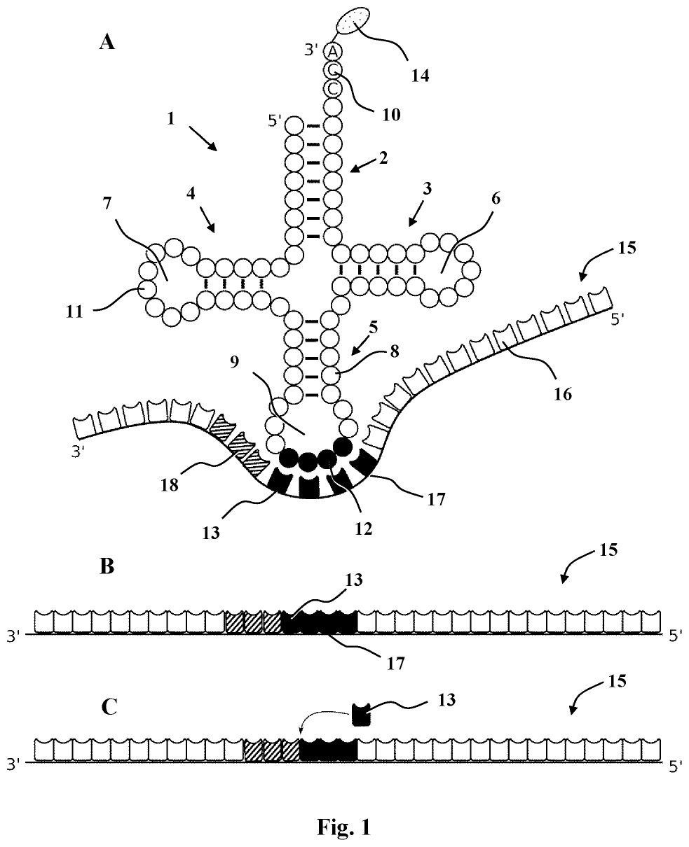

FIG. 1. Schematic example of a synthetic tRNA of the invention and a targeted mRNA for suppressing a +1 frameshift mutation. A. Synthetic tRNA bound to targeted mRNA. B. mRNA with codon carrying insertion. C. Original (wild-type) mRNA without insertion.

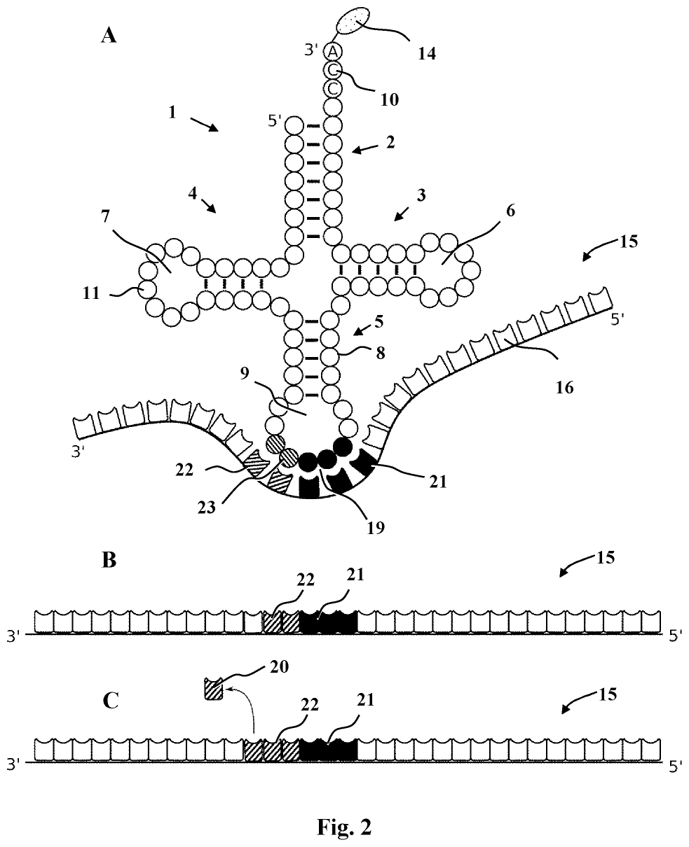

FIG. 2. Schematic example of a synthetic tRNA of the invention and a targeted mRNA for suppressing a −1 frameshift mutation.

FIG. 3. Schematic drawing of a generalized “consensus” tRNA structure and its numbering according to tRNA numbering convention.

FIG. 1 shows a schematic example of a synthetic tRNA 1 of the invention, useful as a +1 frameshift suppressor, and a targeted mRNA 15 carrying a mutated codon 17 with an inserted nucleotide 13. FIG. 1A shows the synthetic tRNA 1 bound to an mRNA 15 having a mutated codon 17 with an inserted nucleotide 13, FIG. 1B shows the mRNA 15 with the mutated codon 17, and Fig. C depicts the original unmutated (wild-type) mRNA 15 receiving the additional nucleotide 13. The synthetic tRNA 1 of the invention is composed of tRNA nucleotides 11 and has the common cloverleaf structure of natural tRNA comprising an acceptor stem 2 with the CCA tail 10, a T arm 3 with the TψC loop 6, a D arm 4 with the D loop 7 and an anticodon arm 5 with a five nucleotide stem portion 8 and the anticodon loop 9. An amino acid 14 is bound to the CCA tail 10 of the acceptor stem 2. The extended anticodon loop 9 consists of nine nucleotides 11 and contains a four-nucleotide (quadruplet) anticodon 12, i.e. a “codon” composed of four nucleotides (solid black circles) instead of the usual three nucleotides. The four-nucleotide anticodon 12 is able to base-pair to a mutated codon 17 (also solid black) on a targeted mRNA 15 composed of mRNA nucleotides 16. The mutated codon 17 carries an insertion, such that the original base triplet codon is extended by the inserted nucleotide 13. The four-nucleotide anticodon 12 (solid black circles) is able to base-pair with the mutated codon 17 (also solid black) on the mRNA 15. The tRNA preferably carries an amino acid encoded by the unmutated codon. A variable loop between the T arm and the anticodon arm is not shown here.

FIG. 2 shows a schematic example of a synthetic tRNA 1 of the invention, useful as a −1 frameshift suppressor. As shown in FIG. 2A, this embodiment of a synthetic tRNA 1 of the invention carries a five-nucleotide anticodon 19, i.e. an anticodon composed of five nucleotides being able to base-pair with complementary five nucleotides on an mRNA 15. FIGS. 2B and 2C show the mutated (B) and the original (C) unmutated mRNA 15. The mutated mRNA 15 carries a mutated codon 22 (hatched) from which a nucleotide 20 has been deleted. The preceding unmutated, i.e. original nucleotide triplet codon, 21 is shown in solid black. In the embodiment shown here, the five-nucleotide anticodon 19 is composed of three nucleotides (solid black circles) being able to base pair to the complementary codon 21 preceding the mutated (truncated) codon 22, and the remaining two nucleotides (hatched circles) are able to base-pair with the mutated codon 22 (also hatched), i.e. with the nucleotide doublet left after the deletion of the nucleotide 20 from the original codon 22. The tRNA 1 may carry an amino acid 14 encoded by the codon 21 preceding the mutated codon 22 or an amino acid 14 encoded by the original unmutated codon 22. It is also possible to load the tRNA with a dipeptide being composed of the amino acids encoded by the codon 21 preceding the mutated codon and the mutated codon. The tRNA could, of course, also be designed to base-pair with two codons where the mutated codon precedes the unmutated codon.

FIG. 3 depicts an example of a tRNA numbered according to the conventional numbering applied to a generalized “consensus” tRNA, beginning with 1 at the 5′ end and ending with 76 at the 3′ end. In such a “consensus” tRNA the nucleotides of the natural anticodon triplet 25 is always at positions 34, 35 and 36, regardless of the actual number of previous nucleotides. A tRNA may, for example, contain additional nucleotides between positions 1 and 34, e.g. in the D loop, and in the variable loop 24 between positions 45 and 46. Additional nucleotides may be numbered with added alphabetic characters, e.g. 20a, 20b etc. In the variable loop 24 the additional nucleotides are numbered with a preceding “e” and a following numeral depending on the position of the nucleotide in the loop. Modified nucleotides, as e.g. listed in Table 1 above, may be present in the sequence.

### EXAMPLE 1

To test the effect of various nucleotides and nucleotide pairs on tRNA stability and ability to read non-canonical codons (nucleotide triplets not encoding proteinogenic amino acids), various constructs have been tested in Hep3B cells. For this, tRNAs have been first transcribed in vitro and then transfected into Hep3B cells along with a reporter plasmid. The reporter plasmid contains a luciferase gene in which downstream of the start codon a non-canonical codon is incorporated to which all tested tRNAs pair. Variations in the tRNA have been performed in the T-stem and anticodon stem, whilst the remaining tRNA body was kept constant.

The following T arms and anticodon arms were tested alone or in combination (see Table 2 below). Only the stem portions were varied, the loop of the T arms (UUCGAAU) and of the anticodon arms (CUUCAAA, anticodon underlined) tested remained the same:

T Arms Tested:

| | | SEQ ID |
|---|---|---|
| Name | Sequence | NO: |
| T stem variant 1 (G51-C63) | GCGGGUUCGAAUCCCGC | 21 |
| T stem variant 2 TS_Glu | CCGGGUUCGAAUCCCGG | 24 |
| T stem variant 3 (C51-G63) | GCCGGUUCGAAUCCGGC | 44 |
| T stem variant 4 (TS2) | AGGCCUUCGAAUGGCCU | 45 |
| T stem variant 5 (A49-G51- | ACGGGUUCGAAUCCCGU | 22 |
| C63-U65 | | |
| T stem variant 6 (G51-C63- | GCGGGUUCGAAUCCCGU | 23 |
| U65) | | |
| Human Ser tRNA (1C) | GCAGGUUCGAAUCCUGC | 43 |

Anticodon arms tested:

| | | SEQ |
|---|---|---|
| Name | Sequence | ID NO: |
| Human Ser tRNA (1C) | AUGGACUUCAAAUCCAU | 46 |
| A-stem variant 1 | AUGGUCUUCAAAACCAU | 47 |
| (U31-A39) | | |

In the combination experiments (modified T stem plus modified Anticodon stem), the above-mentioned A-stem variant 1 (U31-A39) was used as anticodon stem.

T stem and anticodon stem combinations (A/T stem combination) tested:

| | | anticodon |
|---|---|---|
| Name | T-stem | stem |
| A/T stem combination 1 (TA1) | variant 1 | variant 1 |
| A/T stem combination 2 (TGA1) | variant 2 | variant 1 |
| A/T stem combination 3 (BT1) | variant 5 | variant 1 |
| A/T stem combination 4 (BT2) | variant 6 | variant 1 |

In Vitro tRNA Transcription

The template for T7-promoter driven transcription of designed tRNAs was generated by annealing and primer extension of two overlapping DNA oligonucleotides (commercially ordered) covering the whole length of each tRNA including the T7 promoter sequence (5′-TAATACGACTCACTATA-3′). Both oligonucleotides were denatured for 2 min at 95° C. and annealed in their partial overlapping area by incubating for 3 min at room temperature in 0.2 M Tris-HCl (pH 7.5). To fill up the DNA templates to completely double stranded DNA, 0.4 mM dNTPs, 4 U/μL RevertAid Reverse Transcriptase (Thermo Fisher Scientific, USA) and 1×RT buffer were added to the annealed oligonucleotides and reactions were incubated 40 min at 37° C. The DNA template was purified with phenol/chloroform.

For the in vitro T7-driven transcription of tRNAs we mixed 30 μg dsDNA template with 2 mM NTPs, 5 mM GMP and 0.6 U/μL T7 RNA polymerase (Thermo Fisher Scientific, USA) in 1× transcription buffer and incubated overnight at 37° C. tRNAs were precipitated with ethanol, separated on 10% denaturing polyacrylamide gels and eluted overnight at 4° C. with 50 mM KOAc, 200 mM KCl pH 7.0 at constant shaking (1000 rpm). tRNAs were filtered, recovered by ethanol precipitation and resuspended in DEPC-H2O. tRNA integrity was analysed on 10% denaturing polyacrylamide gel electrophoresis and tRNAs were stored at −80° C. for further use.

Testing tRNA Efficiency in Hep3B Cells

Hep3B cells were maintained at 37° C., 5% CO₂ in DMEM media supplemented with 10% foetal bovine serum (FBS) and 1% glutamine (GIBCO). One day prior to co-transfection ˜10,000 cells were seeded in 96-well plates. The transfection mix included the reporter plasmid (pGL4.51 [luc2/CMV/Neo] backbone (Promega, USA)) (25 ng/well), suppressor tRNA (100 ng/well), Lipofectamine 3000 (0.3 μl/well) and P3000 reagent (0.2 μl/well) (Thermo Fisher Scientific, USA) in Opti-MEM (Thermo Fisher Scientific, USA). Cells were incubated with transfection mix for 5 hours. Thereafter, the medium was exchanged with fresh medium. After 24 hours, the cells were washed with 1×PBS and lysed by addition of 1× passive lysis buffer (Promega, USA) under agitation for 15 minutes at room temperature. Luciferase activity was measured by addition of Firefly luciferase reagent (Promega, USA) with the Tecan Spark microplate reader (Tecan, CH).

In the designed tRNA, nucleotide pairs in the T-stem and anticodon stem were varied, whereas all other parts of tRNA remained constant (see above). 100 ng in vitro-transcribed tRNAs was co-transfected with pGL4.51 [luc2/CMV/Neo] expressing the luciferase reporter with the non-canonical codon and expressed for 24 h. The pairing to the non-canonical codon results in luciferase activity which was monitored by determination of the Firefly luciferase expression with a microplate reader. Results of are presented in Table 2 below.

Table 2: Activity of various tRNA designs with optimization in the T or anticodon(A) stems. In the designed tRNA, nucleotide pairs in the T-stem and anticodon stem were varied, whereby all other parts of tRNA remained constant. 100 ng in vitro transcribed tRNAs was co-transfected with pGL4.51 [luc2/CMV/Neo] expressing the luciferase reporter with the non-canonical codon and expressed for 24 h. The efficient pairing to the non-canonical codon results in luciferase activity which was monitored by the firefly luciferase enzymatic activity with a microplate reader. Data are means±SD (n=3).

| | | | % of |
|---|---|---|---|
| | | Firefly luciferase | the Fluc |
| tRNA variant | Sequence changes | activity, RLU/s | activity^{c} |
| T-stem variant 1 | tRNA with T-stem | 10,745 ± 4,873 | 8.6 |
| | changed to increase | | |
| | stability by ΔΔG° = | | |
| | −1.1 kcal/mol | | |
| | (G51-C63) | | |
| T-stem variant 2 | tRNA with T-stem | 4,521 ± 770 | 3.5 |
| | changed to increase | | |
| | stability by ΔΔG° = | | |
| | −0.7 kcal/mol | | |
| | (TS_Glu) | | |
| T-stem variant 3 | tRNA with T-stem | 3,214 ± 2,184 | 2.5 |
| | changed to increase | | |
| | stability by ΔΔG° = | | |
| | −0.6 kcal/mol | | |
| | (C51-G63) | | |
| T-stem variant 4 | tRNA with complete | 624 ± 138 | 0.4 |
| | T-stem changed to | | |
| | increase stability by | | |
| | ΔΔG° = −1.0 | | |
| | kcal/mol (TS2) | | |
| A-stem variant 1 | tRNA with A-stem | 6,383 ± 1,194 | 5.0 |
| | changed (U31-A39) | | |
| Human Ser-tRNA^{a} | Human Ser-tRNA | 1,573 ± 635 | 1.1 |
| | with only exchanged | | |
| | anticodon loop to | | |
| | pair to the non- | | |
| | canonical codon in | | |
| | luciferase (C1) | | |
| FLuc wild type | no tRNA (Fluc) | 123,711 ± 12,608 | 100 |
| (positive control) | | | |
| Fluc with non- | no tRNA (R208X) | 531 ± 71 | 0.3 |
| canonical codon | | | |
| (negative control) | | | |
| Fluc with non- | random tRNA not | 630 ± 275 | 0.4 |
| canonical codon | pairing to the non- | | |
| (negative control) | canonical codon | | |
| | (Q66X 1C) | | |
| Mock transfection | No firefly luciferase, | 128 ± 15 | 0 |
| (negative control)^{b} | no tRNA | | |
| T/A stem | | | |
| combinations | | | |
| A/T-stem | tRNA combination | 111,602 ± 21,914 | 4.6 |
| combination 1 | of T-stem variant 1 | | |
| | and A-stem variant 1 | | |
| | (TA1) | | |
| A/T-stem | tRNA combination | 170,903 ± 37,804 | 8.2 |
| combination 2 | of T-stem variant 2 | | |
| | and A-stem variant 1 | | |
| | (TGA1) | | |
| A/T-stem | tRNA combination | 307,395 ± 52,984 | 13.9 |
| combination 3 | of T-stem variant | | |
| | with ΔΔG° = −1.2 | | |
| | kcal/mol and A-stem | | |
| | variant 1 (BT1) | | |
| A/T-stem | tRNA combination | 130,067 ± 60,487 | 6.2 |
| combination 4^{d} | of T-stem variant | | |
| | with ΔΔG° = −1.6 | | |
| | kcal/mol and A-stem | | |
| | variant 1 (BT2) | | |
| FLuc wild type | no tRNA (Fluc) | 2,059,061 ± 152,899 | 100 |
| (positive control) | | | |
| Fluc with non- | no tRNA (R208X) | 2,756 ± 1,168 | 0.03 |
| canonical codon | | | |
| (negative control) | | | |
| Mock transfection | No firefly luciferase, | 2,116 ± 603 | 0 |
| (negative control) | no tRNA | | |

^{a}The stabilization energy of the T-stem mutants is calculated as a difference (ΔΔG°) to the stabilization energy trough the binding of the natural human Ser-tRNA variant to elongation factor whose energy is ΔG° = 0.5 kcal/mol. The elongation factor specifically binds to the T-stem hence, this energy is only given for T-stem mutants and not for combined mutants.
^{b}Note that sets of the experiments were performed with different luciferase substrates, thus each should be compared to an own set of controls. The two sets are separated by a thick lane.
^{c}The percentage of the Fluc activity was calculated from the wild-type luciferase activity after subtracting the mock values.
^{d}Note that in A/T variant 3 and 4 the T-stem was further mutated to increase the stability^{a}, but those variants were not tested as single T-stem variants.

1. A synthetic transfer RNA comprising an extended anticodon loop having a four-nucleotide anticodon or a five-nucleotide anticodon.

2. The synthetic transfer RNA according to claim 1, wherein the anticodon loop consists of 7 to 12, preferably 7 to 10 or 8 to 10 nucleotides, more preferably 8 or 9 nucleotides.

3. The synthetic transfer RNA according to one of claim 1 or 2, wherein the transfer RNA is aminoacylated.

4. The synthetic transfer RNA according to claim 3, wherein the transfer RNA is aminoacylated with a dipeptide.

5. The synthetic transfer RNA according to one of the preceding claims, wherein the synthetic tRNA has a sequence identity of less than 70%, 65%, 60%, 65% or 50%, preferably of less than 49%, 48%, 47%, 46%, 45%, 44% or 43% to any naturally occurring tRNA.

6. The synthetic transfer RNA according to one of the preceding claims, wherein the synthetic transfer RNA has the same or a higher stability compared to a naturally occurring transfer RNA, whose three-nucleotide anticodon has been replaced with a four-nucleotide anticodon or a five-nucleotide anticodon, or compared to a naturally occurring tRNA for the same cognate amino acid, wherein the stability of a synthetic transfer RNA with a four-nucleotide anticodon is compared with a naturally occurring transfer RNA whose three-nucleotide anticodon has been replaced with a four-nucleotide anticodon or is compared with a naturally occurring tRNA for the same cognate amino acid, and the stability of a synthetic transfer RNA with a five-nucleotide anticodon is compared with a naturally occurring transfer RNA whose three-nucleotide anticodon has been replaced with a five-nucleotide anticodon or is compared with a naturally occurring tRNA for the same cognate amino acid.

7. The synthetic transfer RNA according to one of the preceding claims, wherein the synthetic transfer RNA has a C at position 32 and/or an A at position 37 in the anticodon loop, the numbering following transfer RNA numbering convention.

8. The synthetic transfer RNA according to one of the preceding claims, wherein the extended anticodon loop is flanked by a G-C or C-G pair, or the extended anticodon loop is, in a 5′-3′ direction, flanked by a U-A pair.

9. The synthetic transfer RNA according to one of the preceding claims, the synthetic transfer RNA having or comprising a) a sequence being composed of, in 5′ to 3′ direction, consecutive sequence parts A, B and C, part A having or comprising one of the sequences according to SEQ ID NO: 01, SEQ ID NO: 02, SEQ ID NO: 07, SEQ ID NO: 08, SEQ ID NO: 27, SEQ ID NO: 28, SEQ ID NO: 29 and SEQ ID NO: 30, part B having or comprising one of the sequences according to SEQ ID NO: 03, SEQ ID NO: 04, SEQ ID NO: 06, SEQ ID NO 25, SEQ ID NO: 31 and SEQ ID NO: 32, and part C having or comprising one of the sequences according to SEQ ID NO: 5, SEQ ID NO: 33 and SEQ ID NO: 34, or b) a sequence having at least 90%, preferably at least 95%, 96%, 97%, 98% or 99% sequence identity with one of the sequences according to a) above, or c) a sequence according to one of sequences of a) or b) above, where at least one of the nucleotides is replaced with a corresponding modified nucleotide.

10. The synthetic transfer RNA according to one of the preceding claims for use as a medicament.

11. The synthetic transfer RNA according to one of claims 1 to 9 for use as a medicament in a disease, which is at least partly caused by a frameshift mutation leading to the production of a protein being dysfunctional or non-functional compared to the wild-type protein.

12. The synthetic transfer RNA according to one of claims 1 to 9 for use as a medicament for treating Crohn disease, Tay-Sachs disease, Duchenne muscular dystrophy, cystic fibrosis, neuronal ceroid lipofuscinosis, or neurofibromatosis type 1.


SEQUENCE LISTING (47 sequences)

SEQ ID NO: 1 - RNA, 47 nt, Artificial Sequence
  Part A 4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“extended (4nt) anticodon”
  misc_feature 43..45: /note=“n = A, C, G or U, or any modified base”
  misc_feature 47: /note=“n = A, C, G or U, or any modified base”
nncagnnugnncgagnngucuaagnnnnnngccunnnnaugcnnnun

SEQ ID NO: 2 - RNA, 46 nt, Artificial Sequence
  Part A 4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“extended (4nt) anticodon”
  misc_feature 42..44: /note=“n = A, C, G or U, or any modified base”
  misc_feature 46: /note=“n = A, C, G or U, or any modified base”
nncagnnugnncgagnngucuaagnnnnnngccunnnnagcnnnun

SEQ ID NO: 3 - RNA, 11 nt, Artificial Sequence
  Part B variable loop
  misc_feature 1..11: /note=“n = A, C, G or U, or any modified base”
nnnnnnnnnnn

SEQ ID NO: 4 - RNA, 15 nt, Artificial Sequence
  Part B variable loop
  misc_feature 1..15: /note=“n = A, C, G or U, or any modified base”
nnnnnnnnnnnnnnn

SEQ ID NO: 5 - RNA, 29 nt, Artificial Sequence
  Part C
  misc_feature 2: /note=“n = A, C, G or U, or any modified base”
  misc_feature 6: /note=“n = A, C, G or U, or any modified base”
  misc_feature 14: /note=“n = A, C, G or U, or any modified base”
  misc_feature 18..20: /note=“n = A, C, G or U, or any modified base”
  misc_feature 24..25: /note=“n = A, C, G or U, or any modified base”
cnuggnuucgaaunccannncugnnacca

SEQ ID NO: 6 - RNA, 15 nt, Artificial Sequence
  Part B variable loop
uggggucacuccccg

SEQ ID NO: 7 - RNA, 48 nt, Artificial Sequence
  Part A 5 nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 44..46: /note=“n = A, C, G or U, or any modified base”
  misc_feature 48: /note=“n = A, C, G or U, or any modified base”
nncagnnugnncgagnngucuaagnnnnnngccunnnnnaugcnnnun

SEQ ID NO: 8 - RNA, 47 nt, Artificial Sequence
  Part A 5 nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 43..45: /note=“n = A, C, G or U, or any modified base”
  misc_feature 47: /note=“n = A, C, G or U, or any modified base”
nncagnnugnncgagnngucuaagnnnnnngccunnnnnagcnnnun

SEQ ID NO: 9 - RNA, 87 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“4nt anticodon”
  misc_feature 43..45: /note=“n = A, C, G or U, or any modified base”
  misc_feature 47..58: /note=“n = A, C, G or U, or any modified base”
  misc_feature 60: /note=“n = A, C, G or U, or any modified base”
  misc_feature 64: /note=“n = A, C, G or U, or any modified base”
  misc_feature 72: /note=“n = A, C, G or U, or any modified base”
  misc_feature 76..78: /note=“n = A, C, G or U, or any modified base”
  misc_feature 82..83: /note=“n = A, C, G or U, or any modified base”
nncagnnugnncgagnngucuaagnnnnnngccunnnnaugcnnnunnnnnnnnnnnncnuggnuucgaaunccannncugnnacca

SEQ ID NO: 10 - RNA, 86 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“4nt anticodon”
  misc_feature 42..44: /note=“n = A, C, G or U, or any modified base”
  misc_feature 46..57: /note=“n = A, C, G or U, or any modified base”
  misc_feature 59: /note=“n = A, C, G or U, or any modified base”
  misc_feature 63: /note=“n = A, C, G or U, or any modified base”
  misc_feature 71: /note=“n = A, C, G or U, or any modified base”
  misc_feature 75..77: /note=“n = A, C, G or U, or any modified base”
  misc_feature 81..82: /note=“n = A, C, G or U, or any modified base”
nncagnnugnncgagnngucuaagnnnnnngccunnnnagcnnnunnnnnnnnnnnncnuggnuucgaaunccannncugnnacca

SEQ ID NO: 11 - RNA, 91 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“4nt anticodon”
  misc_feature 43..45: /note=“n = A, C, G or U, or any modified base”
  misc_feature 47: /note=“n = A, C, G or U, or any modified base”
  misc_feature 64: /note=“n = A, C, G or U, or any modified base”
  misc_feature 68: /note=“n = A, C, G or U, or any modified base”
  misc_feature 76: /note=“n = A, C, G or U, or any modified base”
  misc_feature 80..82: /note=“n = A, C, G or U, or any modified base”
  misc_feature 86..87: /note=“n = A, C, G or U, or any modified base”
nncagnnugnncgagnngucuaagnnnnnngccunnnnaugcnnnunuggggucacuccccgcnuggnuucgaaunccannncugnnacca

SEQ ID NO: 12 - RNA, 90 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base”
  misc_feature 35..38: /note=“4nt anticodon”
  misc_feature 42..44: /note=“n = A, C, G or U, or any modified base”
  misc_feature 46: /note=“n = A, C, G or U, or any modified base”
  misc_feature 63: /note=“n = A, C, G or U, or any modified base”
  misc_feature 67: /note=“n = A, C, G or U, or any modified base”
  misc_feature 75: /note=“n = A, C, G or U, or any modified base”
  misc_feature 79..81: /note=“n = A, C, G or U, or any modified base”
  misc_feature 85..86: /note=“n = A, C, G or U, or any modified base”
nncagnnugnncgagnngucuaagnnnnnngccunnnnagcnnnunuggggucacuccccgcnuggnuucgaaunccannncugnnacca

SEQ ID NO: 13 - RNA, 88 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 44..46: /note=“n = A, C, G or U, or any modified base”
  misc_feature 48..59: /note=“n = A, C, G or U, or any modified base”
  misc_feature 61: /note=“n = A, C, G or U, or any modified base”
  misc_feature 65: /note=“n = A, C, G or U, or any modified base”
  misc_feature 73: /note=“n = A, C, G or U, or any modified base”
  misc_feature 77..79: /note=“n = A, C, G or U, or any modified base”
  misc_feature 83..84: /note=“n = A, C, G or U, or any modified base”
nncagnnugnncgagnngucuaagnnnnnngccunnnnnaugcnnnunnnnnnnnnnnncnuggnuucgaaunccannncugnnacca

SEQ ID NO: 14 - RNA, 87 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 43..45: /note=“n = A, C, G or U, or any modified base”
  misc_feature 47..58: /note=“n = A, C, G or U, or any modified base”
  misc_feature 60: /note=“n = A, C, G or U, or any modified base”
  misc_feature 64: /note=“n = A, C, G or U, or any modified base”
  misc_feature 72: /note=“n = A, C, G or U, or any modified base”
  misc_feature 76..78: /note=“n = A, C, G or U, or any modified base”
  misc_feature 82..83: /note=“n = A, C, G or U, or any modified base”
nncagnnugnncgagnngucuaagnnnnnngccunnnnnagcnnnunnnnnnnnnnnncnuggnuucgaaunccannncugnnacca

SEQ ID NO: 15 - RNA, 92 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 44..46: /note=“n = A, C, G or U, or any modified base”
  misc_feature 48: /note=“n = A, C, G or U, or any modified base”
  misc_feature 65: /note=“n = A, C, G or U, or any modified base”
  misc_feature 69: /note=“n = A, C, G or U, or any modified base”
  misc_feature 77: /note=“n = A, C, G or U, or any modified base”
  misc_feature 81..83: /note=“n = A, C, G or U, or any modified base”
  misc_feature 87..88: /note=“n = A, C, G or U, or any modified base”
nncagnnugnncgagnngucuaagnnnnnngccunnnnnaugcnnnunuggggucacuccccgcnuggnuucgaaunccannncugnnacca

SEQ ID NO: 16 - RNA, 91 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base”
  misc_feature 35..39: /note=“5nt anticodon”
  misc_feature 43..45: /note=“n = A, C, G or U, or any modified base”
  misc_feature 47: /note=“n = A, C, G or U, or any modified base”
  misc_feature 64: /note=“n = A, C, G or U, or any modified base”
  misc_feature 68: /note=“n = A, C, G or U, or any modified base”
  misc_feature 76: /note=“n = A, C, G or U, or any modified base”
  misc_feature 80..82: /note=“n = A, C, G or U, or any modified base”
  misc_feature 86..87: /note=“n = A, C, G or U, or any modified base”
nncagnnugnncgagnngucuaagnnnnnngccunnnnnagcnnnunuggggucacuccccgcnuggnuucgaaunccannncugnnacca

SEQ ID NO: 17 - RNA, 18 nt, Artificial Sequence
  U31-A39 4nt anticodon arm
  misc_feature 8..11: /note=“n = A, C, G or U, or any modified base” /note=“4nt anticodon”
auggucunnnnaaaccau

SEQ ID NO: 18 - RNA, 19 nt, Artificial Sequence
  U31-A39 5nt anticodon arm
  misc_feature 8..12: /note=“n = A, C, G or U, or any modified base” /note=“5nt anticodon”
auggucunnnnnaaaccau

SEQ ID NO: 19 - RNA, 17 nt, Artificial Sequence
  T arm consensus 1
  misc_feature 1: /note=“n = A, G or C, or any corresponding modified nucleotide”
  misc_feature 6..12: /note=“n = A, C, G or U, or any modified base” /note=“T-Loop”
  misc_feature 17: /note=“n = C, U or G, or any corresponding modified nucleotide”
ncgggnnnnnnncccgn

SEQ ID NO: 20 - RNA, 17 nt, Artificial Sequence
  T arm consensus 2
  misc_feature 1: /note=“n = A, G or C, or any corresponding modified nucleotide”
  misc_feature 6..12: /note=“T-Loop”
  misc_feature 17: /note=“n = C, U or G, or any corresponding modified nucleotide”
ncggguucgaaucccgn

SEQ ID NO: 21 - RNA, 17 nt, Artificial Sequence
  T arm with T stem variant 1 (G51-C63)
  misc_feature 6..12: /note=“T-Loop”
gcggguucgaaucccgc

SEQ ID NO: 22 - RNA, 17 nt, Artificial Sequence
  T arm with T stem variant 5
  misc_feature 6..12: /note=“T-Loop”
acggguucgaaucccgu

SEQ ID NO: 23 - RNA, 17 nt, Artificial Sequence
  T arm with T stem variant 6
  misc_feature 6..12: /note=“T-Loop”
gcggguucgaaucccgu

SEQ ID NO: 24 - RNA, 17 nt, Artificial Sequence
  T arm with T stem variant 2 (Ts_Glu)
  misc_feature 6..12: /note=“T-Loop”
ccggguucgaaucccgg

SEQ ID NO: 25 - RNA, 17 nt, Artificial Sequence
  Variable loop
  misc_feature 6..11: /note=“n = A, C, G or U, or any modified base”
uggggnnnnnnccccgc

SEQ ID NO: 26 - RNA, 17 nt, Artificial Sequence
  variable loop
ugggguccacuccccgc

SEQ ID NO: 27 - RNA, 47 nt, Artificial Sequence
  Part A 4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base” /note=“4nt anticodon”
  misc_feature 43..45: /note=“n = A, C, G or U, or any modified base”
  misc_feature 47: /note=“n = A, C, G or U, or any modified base”
nncagnnugnncgagnngucuaagnnnnnngucunnnnaaacnnnun

SEQ ID NO: 28 - RNA, 47 nt, Artificial Sequence
  Part A 4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..27: /note=“n = A, C, G or U, or any modified base”
  misc_feature 35..38: /note=“n = A, C, G or U, or any modified base” /note=“4nt anticodon”
  misc_feature 47: /note=“n = A, C, G or U, or any modified base”
nncagnnugnncgagnngucuaagnnnauggucunnnnaaaccauun

SEQ ID NO: 29 - RNA, 48 nt, Artificial Sequence
  Part A 5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base” /note=“5nt anticodon”
  misc_feature 44..46: /note=“n = A, C, G or U, or any modified base”
  misc_feature 48: /note=“n = A, C, G or U, or any modified base”
nncagnnugnncgagnngucuaagnnnnnngucunnnnnaaacnnnun

SEQ ID NO: 30 - RNA, 48 nt, Artificial Sequence
  Part A 5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..27: /note=“n = A, C, G or U, or any modified base”
  misc_feature 35..39: /note=“n = A, C, G or U, or any modified base” /note=“5nt anticodon”
  misc_feature 48: /note=“n = A, C, G or U, or any modified base”
nncagnnugnncgagnngucuaagnnnauggucunnnnnaaaccauun

SEQ ID NO: 31 - RNA, 17 nt, Artificial Sequence
  Part B variable loop
  misc_feature 1..17: /note=“n = A, C, G or U, or any modified base”
nnnnnnnnnnnnnnnnn

SEQ ID NO: 32 - RNA, 17 nt, Artificial Sequence
  Part B variable loop
ugggguccacuccccgc

SEQ ID NO: 33 - RNA, 29 nt, Artificial Sequence
  Part C
  misc_feature 2: /note=“n = A, C, G or U, or any modified base”
  misc_feature 7..13: /note=“n = A, C, G or U, or any modified base”
  misc_feature 18..20: /note=“n = A, C, G or U, or any modified base”
  misc_feature 24..25: /note=“n = A, C, G or U, or any modified base”
cncgggnnnnnnncccgnnncugnnacca

SEQ ID NO: 34 - RNA, 29 nt, Artificial Sequence
  Part C
  misc_feature 2: /note=“n = A, C, G or U, or any modified base”
  misc_feature 18..20: /note=“n = A, C, G or U, or any modified base”
  misc_feature 24..25: /note=“n = A, C, G or U, or any modified base”
cncggguucgaaucccgnnncugnnacca

SEQ ID NO: 35 - RNA, 94 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base” /note=“4nt anticodon”
  misc_feature 43..45: /note=“n = A, C, G or U, or any modified base”
  misc_feature 47..65: /note=“n = A, C, G or U, or any modified base”
  misc_feature 67: /note=“n = A, C, G or U, or any modified base”
  misc_feature 72..78: /note=“n = A, C, G or U, or any modified base”
  misc_feature 83..85: /note=“n = A, C, G or U, or any modified base”
  misc_feature 89..90: /note=“n = A, C, G or U, or any modified base”
nncagnnugnncgagnngucuaagnnnnnngucunnnnaaacnnnunnnnnnnnnnnnnnnnnnncncgggnnnnnnncccgnnncugnnacca

SEQ ID NO: 36 - RNA, 94 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base” /note=“4nt anticodon”
  misc_feature 43..45: /note=“n = A, C, G or U, or any modified base”
  misc_feature 47: /note=“n = A, C, G or U, or any modified base”
  misc_feature 67: /note=“n = A, C, G or U, or any modified base”
  misc_feature 72..78: /note=“n = A, C, G or U, or any modified base”
  misc_feature 83..85: /note=“n = A, C, G or U, or any modified base”
  misc_feature 89..90: /note=“n = A, C, G or U, or any modified base”
nncagnnugnncgagnngucuaagnnnnnngucunnnnaaacnnnunugggguccacucccccgccncgggnnnnnnncccgnnncugnnacca

SEQ ID NO: 37 - RNA, 94 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..38: /note=“n = A, C, G or U, or any modified base” /note=“4nt anticodon”
  misc_feature 43..45: /note=“n = A, C, G or U, or any modified base”
  misc_feature 47: /note=“n = A, C, G or U, or any modified base”
  misc_feature 67: /note=“n = A, C, G or U, or any modified base”
  misc_feature 83..85: /note=“n = A, C, G or U, or any modified base”
  misc_feature 89..90: /note=“n = A, C, G or U, or any modified base”
nncagnnugnncgagnngucuaagnnnnnngucunnnnaaacnnnunugggguccacucccccgccncggguucgaaucccgnnncugnnacca

SEQ ID NO: 38 - RNA, 94 nt, Artificial Sequence
  4nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..27: /note=“n = A, C, G or U, or any modified base”
  misc_feature 35..38: /note=“n = A, C, G or U, or any modified base” /note=“4nt anticodon”
  misc_feature 43..45: /note=“n = A, C, G or U, or any modified base”
  misc_feature 47: /note=“n = A, C, G or U, or any modified base”
  misc_feature 67: /note=“n = A, C, G or U, or any modified base”
  misc_feature 83..85: /note=“n = A, C, G or U, or any modified base”
  misc_feature 89..90: /note=“n = A, C, G or U, or any modified base”
nncagnnugnncgagnngucuaagnnnauggucunnnnaaacnnnunugggguccacucccccgccncggguucgaaucccgnnncugnnacca

SEQ ID NO: 39 - RNA, 95 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base” /note=“5nt anticodon”
  misc_feature 44..46: /note=“n = A, C, G or U, or any modified base”
  misc_feature 48-66: /note=“n = A, C, G or U, or any modified base”
  misc_feature 68: /note=“n = A, C, G or U, or any modified base”
  misc_feature 73..79: /note=“n = A, C, G or U, or any modified base”
  misc_feature 84..86: /note=“n = A, C, G or U, or any modified base”
  misc_feature 90..91: /note=“n = A, C, G or U, or any modified base”
nncagnnugnncgagnngucuaagnnnnnngucunnnnnaaacnnnunnnnnnnnnnnnnnnnnnncncgggnnnnnnncccgnnncugnnacca

SEQ ID NO: 40 - RNA, 95 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base” /note=“5nt anticodon”
  misc_feature 44..46: /note=“n = A, C, G or U, or any modified base”
  misc_feature 48: /note=“n = A, C, G or U, or any modified base”
  misc_feature 68: /note=“n = A, C, G or U, or any modified base”
  misc_feature 73..79: /note=“n = A, C, G or U, or any modified base”
  misc_feature 84..86: /note=“n = A, C, G or U, or any modified base”
  misc_feature 90..91: /note=“n = A, C, G or U, or any modified base”
nncagnnugnncgagnngucuaagnnnnnngucunnnnnaaacnnnunugggguccacucccccgccncgggnnnnnnncccgnnncugnnacca

SEQ ID NO: 41 - RNA, 95 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..30: /note=“n = A, C, G or U, or any modified base”
  misc_feature 35..39: /note=“n = A, C, G or U, or any modified base” /note=“5nt anticodon”
  misc_feature 44..46: /note=“n = A, C, G or U, or any modified base”
  misc_feature 48: /note=“n = A, C, G or U, or any modified base”
  misc_feature 68: /note=“n = A, C, G or U, or any modified base”
  misc_feature 84..86: /note=“n = A, C, G or U, or any modified base”
  misc_feature 90..91: /note=“n = A, C, G or U, or any modified base”
nncagnnugnncgagnngucuaagnnnnnngucunnnnnaaacnnnunugggguccacucccccgccncggguucgaaucccgnnncugnnacca

SEQ ID NO: 42 - RNA, 95 nt, Artificial Sequence
  5nt anticodon tRNA
  misc_feature 1..2: /note=“n = A, C, G or U, or any modified base”
  misc_feature 6..7: /note=“n = A, C, G or U, or any modified base”
  misc_feature 10..11: /note=“n = A, C, G or U, or any modified base”
  misc_feature 16..17: /note=“n = A, C, G or U, or any modified base”
  misc_feature 25..27: /note=“n = A, C, G or U, or any modified base”
  misc_feature 35..39: /note=“n = A, C, G or U, or any modified base” /note=“5nt anticodon”
  misc_feature 44..46: /note=“n = A, C, G or U, or any modified base”
  misc_feature 48: /note=“n = A, C, G or U, or any modified base”
  misc_feature 68: /note=“n = A, C, G or U, or any modified base”
  misc_feature 84..86: /note=“n = A, C, G or U, or any modified base”
  misc_feature 90..91: /note=“n = A, C, G or U, or any modified base”
nncagnnugnncgagnngucuaagnnnauggucunnnnnaaacnnnunugggguccacucccccgccncggguucgaaucccgnnncugnnacca

SEQ ID NO: 43 - RNA, 17 nt, Homo sapiens
  T arm tRNA Ser (1C)
  misc_feature 6..12: /note=“T-Loop”
gcagguucgaauccugc

SEQ ID NO: 44 - RNA, 17 nt, Artificial Sequence
  T arm with T stem variant 3 (C51-G63)
  misc_feature 6..12: /note=“T-Loop”
gccgguucgaauccggc

SEQ ID NO: 45 - RNA, 17 nt, Artificial Sequence
  T arm with T stem variant 4 (TS2)
  misc_feature 6..12: /note=“T-Loop”
aggccuucgaauggccu

SEQ ID NO: 46 - RNA, 17 nt, Artificial Sequence
  Anticodon arm tRNA Ser
auggacuucaaauccau

SEQ ID NO: 47 - RNA, 17 nt, Artificial Sequence
  Anticodon arm U31-A39
auggucuucaaaaccau